(19) United States
(12) Patent Application Publication
(10) Pub. No.: US 2012/0149586 A1
(43) Pub. Date: Jun. 14, 2012

(54) METHODS OF PREDICTING THE RISK OF RECURRENCE OF CANCER

(75) Inventor: Glen Weiss, Phoenix, AZ (US)

(73) Assignees: SCOTTSDALE HEALTHCARE, Scottsdale, AZ (US); THE TRANSLATIONAL GENOMICS RESEARCH INSTITUTE, Phoenix, AZ (US)

(21) Appl. No.: 13/057,739

(22) PCT Filed: Aug. 14, 2009
(86) PCT No.: PCT/US09/53876

§ 371 Date: Jan. 12, 2012

(51) Int. Cl. C12Q 1/68; C07H 21/04; G01N 21/64; C40B 30/00

(52) U.S. Cl. 506/7; 435/6.12; 536/24.31; 435/6.11

(57) ABSTRACT

Disclosed herein are methods of predicting the risk of recurrence of cancer on the basis of the expression of microRNA of the miR-520 family.

### CROSS REFERENCE TO RELATED APPLICATION

This application claims the benefit of U.S. Provisional Application 61/089,431 filed 15 Aug. 2008.

### STATEMENT REGARDING FEDERALLY SPONSORED RESEARCH

The US government retains certain rights in this invention as provided by the terms of grant number P50-58187 awarded by the National Cancer Institute and U01-CA85070 awarded by the National Institutes of Health.

### BACKGROUND OF THE INVENTION

Lung carcinoma remains the leading cause of cancer death worldwide for both men and women. Biomarkers that signal early detection, prognosis, therapeutic response, recurrence free survival and overall survival are needed.

### BRIEF SUMMARY OF THE INVENTION

The present invention provides among other things: a method of predicting recurrence of non-small cell lung cancer.

It is an object of the invention identify non-small cell lung cancer patients with an increased risk of recurrence that would be otherwise predicted based on existing clinical or pathologic risk factors such as stage of disease, type of surgical intervention, stage, and grade among others.

It is an object of the invention to provide a blood test to identify non-small cell lung cancer patients with an increased risk of recurrence.

The above and other objects may be achieved using methods involving obtaining a sample from a subject, assessing the expression of one or more targets from the miR-520 family (alone or in combination), and comparing the expression of the target to a level of expression predetermined to correlate with a reduced risk of recurrence of non-small cell lung cancer. The sample may include a tumor cell such as a sputum sample or biopsy, or include serum such as a whole blood sample. If the sample includes a tumor cell, the tumor cell may have a chromosomal aberration in 19q13. Expression may be assessed by any method that accurately measures miR expression, including microarray, RTPCR based methods, or direct sequencing of cDNA derived from the miRNA. A reduced risk of recurrence may be any method of measuring reduced risk, including disease free survival and overall survival. If the risk is overall survival, the stage of the disease may be assessed as well. If so, then the stage may be Stage II or Stage III alone or in combination. The expression level may be any detectable level of expression, but may include the median expression level in a sample group comprising at least 100 small cell lung cancer tumors, a level of dye incorporation that yields a preset optical density within 40 PCR cycles if RTPCR based methods are used, or a level of 1×10⁻⁵ that of expression of a housekeeping gene.

The above and other objects may be achieved using kits that comprise a first nucleic acid that hybridizes to a target in the miR-520e family. The first nucleic acid may include any fluorescent label that allows the nucleic acid to be detected. The first nucleic acid may be affixed to a solid substrate. In some aspects of the invention, the kit comprises a second nucleic acid that hybridizes to a target in the miR-520e family, but does not overlap the first nucleic acid. This kit may also include a reverse transcriptase and/or a DNA polymerase.

### BRIEF DESCRIPTION OF THE FIGURES

A more complete understanding of the present invention may be derived by referring to the detailed description when considered in connection with the following illustrative figures.

FIG. 1 depicts progression free survival and overall survival based on miR-520e expression in all pathologic stages combined.

FIG. 2 depicts the growth of H2122 cells in the presence of miR-520e mimic and miR-520e inhibitor.

FIG. 3 depicts the growth of H157 cells in the presence of miR-520e mimic and miR-520e inhibitor.

FIG. 4 depicts the growth of colo699 cells in the presence of miR-520e mimic and miR-520e inhibitor.

FIG. 5 depicts progression free survival and overall survival based on miR-520e expression in a set of samples that only display Stage I disease.

FIG. 6 depicts progression free survival and overall survival based on miR-520e expression in a set of samples that display both Stage I and Stage II disease.

FIG. 7 depicts progression free survival and overall survival based on miR-520e expression in a set of samples that only display Stage II disease.

FIG. 8 depicts progression free survival and overall survival based on miR-520e expression in a set of samples that display both Stage II and Stage III disease.

FIG. 9 depicts progression free survival and overall survival based on miR-520e expression in a set of samples that only display Stage III disease.

FIG. 10 depicts progression free survival and overall survival on the basis of type of surgery performed.

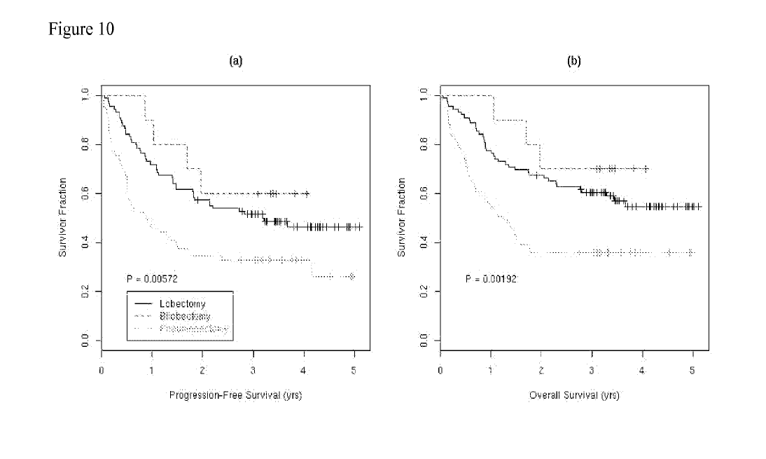

FIG. 11 depicts progression free survival and overall survival on the basis of disease stage.

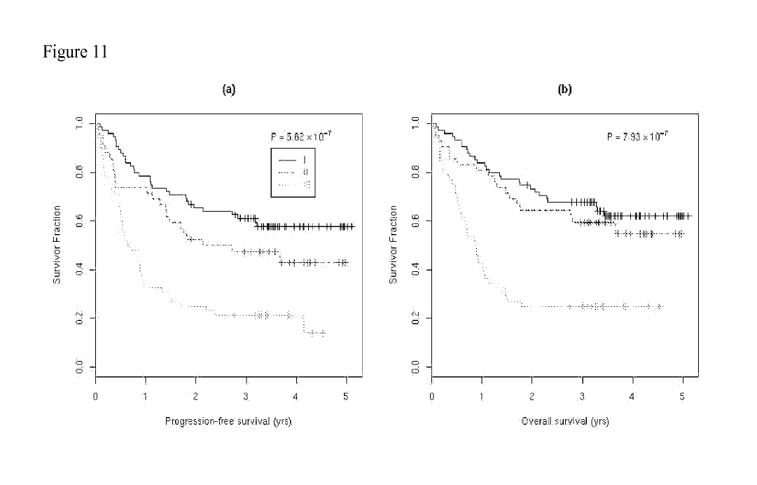

FIG. 12 depicts progression free survival and overall survival on the basis of histological subtypes.

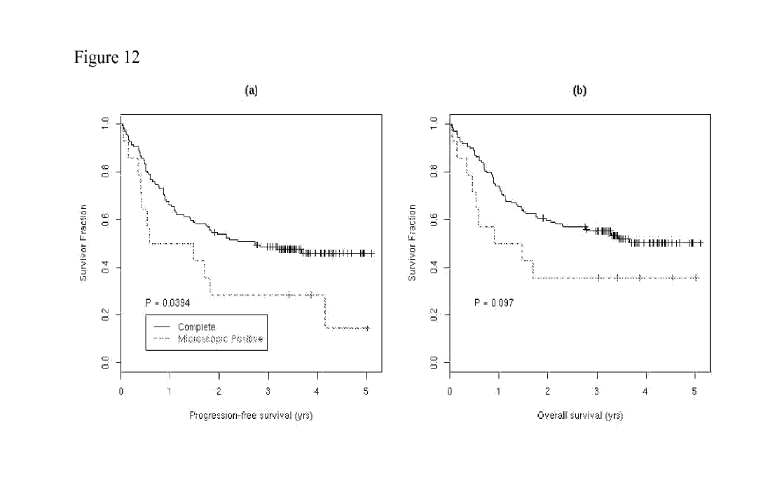

### DETAILED DESCRIPTION OF THE INVENTION

A target includes any molecular structure produced by a cell, expressed inside the cell, accessible on the cell surface, or secreted by the cell. Targets include proteins, lipids, carbohydrates, nucleic acids including RNA molecules and genomic DNA sequences, subcellular structures, glycoproteins, viruses and any other such structure now known or yet to be disclosed whether alone or in combination.

A specific target may be identified by the sequence of a nucleic acid from which it can be derived. Examples of such nucleic acids include miRNA, tRNA, siRNA, mRNA, cDNA, or genomic DNA sequences. Alternatively, a specific target may be identified by a protein sequence. However, the specific target is not limited to the products of the exact nucleic acid sequence or protein sequence by which it may be identified. Rather, a specific target encompasses all sequences that yield positive expression when the expression of the specific target is assessed. Examples of sequences encompassed by a specific target identified by a nucleic acid molecule include point mutations, silent mutations, deletions, frameshift mutations, translocations, alternative splicing derivatives, differentially methylated sequences, differentially modified protein sequences, and any other variation that results in a product that may be identified as the specific target. The following nonlimiting example is included for the purposes of clarifying this concept: if expression of a specific target in a sample is assessed by RTPCR, and if the sample expresses a sequence different from the sequence used to identify the specific target (e.g. a variation of one or more nucleic acid bases,) but detectable expression of the target is still determined, then the target encompasses the sequence present in the sample.

Expression encompasses any and all processes through which material derived from a nucleic acid template may be produced. Expression thus includes RNA transcription, mRNA splicing, protein translation, protein folding, post-translational modification, membrane transport, associations with other molecules, addition of carbohydrate moeties to proteins, phosphorylation, protein complex formation and any other process along a continuum that results in biological material derived from genetic material. Expression also encompasses all processes through which the production of material derived from a nucleic acid template may be actively or passively suppressed. Such processes include all aspects of transcriptional and translational regulation. Examples include heterochromatic silencing, transcription factor inhibition, any form of RNAi silencing, microRNA silencing, alternative splicing, protease digestion, posttranslational modification, and alternative protein folding.

Expression may be assessed by any number of methods used to detect material derived from a nucleic acid template used currently in the art and yet to be developed. Examples of such methods include any nucleic acid detection method including the following nonlimiting examples, microarray analysis, RNA in situ hybridization, RNAse protection assay, Northern blot, reverse transcriptase PCR, quantitative PCR, quantitative reverse transcriptase PCR, quantitative real-time reverse transcriptase PCR, reverse transcriptase treatment followed by direct sequencing, or any other method of detecting a specific nucleic acid now known or yet to be disclosed. Other examples include any process of assessing expression that uses an antibody including the following nonlimiting examples, flow cytometry, immunohistochemistry, ELISA, Western blot, and immunoaffinity chromatography. Antibodies may be monoclonal, polyclonal, or any antibody fragment including an Fab, F(ab)₂, Fv, scFv, phage display antibody, peptibody, multispecific ligand, or any other reagent with specific binding to a target. Such methods also include direct methods used to assess protein expression including the following nonlimiting examples: HPLC, mass spectrometry, protein microarray analysis, PAGE analysis, isoelectric focusing, 2-D gel electrophoresis, and enzymatic assays. Samples from which expression may be detected include single cells, whole organs or any fraction of a whole organ, whether in vitro, ex vivo, in vivo, or post-mortem.

Other methods used to assess expression include the use of natural or artificial ligands capable of specifically binding a target, including a protein, carbohydrate, fat, nucleic acid, catalytic site, or any combination of these such as an enzyme, glycoprotein, cell membrane, virus, cell, organ, organelle, or any uni- or multimolecular structure that constitutes a target that may be specifically bound by a ligand. Such ligands include antibodies, antibody complexes, conjugates, natural ligands, small molecules, nanoparticles, or any other molecular entity capable of specific binding to a target. Ligands may be associated with a label such as a radioactive isotope or chelate thereof, dye (fluorescent or nonfluorescent,) stain, enzyme, metal, or any other substance capable of aiding a machine or a human eye from differentiating a cell expressing a target from a cell not expressing a target. Additionally, expression may be assessed by monomeric or multimeric ligands associated with substances capable of killing the cell. Such substances include protein or small molecule toxins, cytokines, pro-apoptotic substances, pore forming substances, radioactive isotopes, or any other substance capable of killing a cell.

Positive expression encompasses any difference between a cell expressing a specific target and a cell that does not express a specific target. The exact nature of positive expression varies by the method, but is well known to those skilled in the art of practicing a particular method. Positive expression may be assessed by a detector, an instrument containing a detector, or by aided or unaided human eye. Examples include but are not limited to specific staining of cells expressing a target in an IHC slide, binding of RNA from a sample to a microarray and detection of binding through the use of said microarray, a particular rate of dye incorporation in realtime RTPCR measured in ΔCt or alternatively in the number of PCR cycles necessary to reach a particular optical density at a wavelength at which a double stranded DNA binding dye (e.g. SYBR Green) incorporates, through release of label from a previously labeled reporter probe used in a real-time RTPCR reaction, detection of fluorescence on a cell expressing a target by a flow cytometer, the presence of radiolabeled bands on film in a Northern blot, detection of labeled blocked RNA by RNAse protection assay, cell death measured by apoptotic markers, cell death measured by shrinkage of a tumor, or any other signal for the expression of a target in existence now or yet to be developed. In some aspects of the invention, positive expression is a sufficient level of expression to correlate with a particular response such as susceptibility to cancer recurrence.

In some aspects of the invention, reduced expression constitutes no detectable expression. However, the concept of reduced expression further encompasses insufficient expression to reach or exceed a threshold, cutoff, or level that has been previously shown to result in a particular cellular or physiological response. Reduced expression may include similar expression relative to a control that has been previously determined not to express the target or similar expression to a control that has been previously determined not to exhibit the response. In this case, even though expression may be detectable, it still constitutes reduced expression. In some aspects of the invention, an expression level of a target in a control known to have a reduced risk of recurrence is predetermined and expression similar to that level is correlated with reduced risk of recurrence. Reduced expression includes expression that is 75% 50%, 25%, 10%, 5%, 1%, 0.1%, or less of that of a control cell or a median level of expression in a population. Reduced expression may also include less than 1×10⁻⁵ or less expression normalized to the expression of a housekeeping gene.

The invention contemplates assessing the expression of the target in any biological sample from which the expression may be assessed. One skilled in the art would know to select a particular biological sample and how to collect said sample depending upon the target that is being assessed. Examples of sources of samples include but are not limited to biopsy or other in vivo or ex vivo analysis of prostate, breast, skin, muscle, facia, brain, endometrium, lung, head and neck, pancreas, small intestine, blood, liver, testes, ovaries, colon, skin, stomach, esophagus, spleen, lymph node, bone marrow, kidney, placenta, or fetus. In some aspects of the invention, the sample comprises a fluid sample, such as peripheral blood, lymph fluid, ascites, serous fluid, pleural effusion, sputum, cerebrospinal fluid, amniotic fluid, lacrimal fluid, stool, or urine. In one aspect of the invention, the sample comprises primary or metastatic NSCLC cells. In another, oit comprises sputum. In another aspect of the invention, the sample comprises blood. MicroRNA is readily detectable in blood and blood compartments such as serum or plasma or whole blood by any of a number of methods. See, for example, Chen X et al, Cell Research 18 983-984, October 2008.

Assessing the risk of a particular disease outcome includes the performing of any type of test, assay, examination, result, readout, or interpretation that correlates with an increased or decreased probability that an individual has had, currently has, or will develop a particular disease, disorder, symptom, syndrome, or any condition related to health or bodily state. Examples of disease outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a particular disease encompasses diagnosis in which the type of disease afflicting a subject is determined. Assessing the risk of a disease outcome also encompasses the concept of prognosis. A prognosis may be any assessment of the risk of disease outcome in an individual in which a particular disease has been diagnosed. Assessing the risk further encompasses prediction of therapeutic response in which a treatment regimen is chosen based on the assessment. Assessing the risk also encompasses a prediction of overall survival after diagnosis.

A subject includes any human or non-human mammal, including for example: a primate, cow, horse, pig, sheep, goat, dog, cat, or rodent, capable of developing lung cancer including human patients that are suspected of having endometrial cancer, that have been diagnosed with lung cancer, or that have a family history of lung cancer. Methods of identifying subjects suspected of having lung cancer include but are not limited to: physical examination, family medical history, subject medical history, endometrial biopsy, or a number of imaging technologies such as ultrasonography, computed tomography, magnetic resonance imaging, magnetic resonance spectroscopy, or positron emission tomography.

MicroRNA's (miR's) include non-coding RNA's 18 to 36 nucleotides in length that inhibit gene expression by binding to a sequence complementary to the miR sequence, often located in the 3′ untranslated region (UTR) of the target mRNA. Mechanisms of gene silencing include repression of protein translation and downregulation of protein expression. The concept of miR's includes non-nucleotide small molecule compositions of matter that are capable of specifically binding the 3′ UTR of one or more genes, thereby silencing the expression of those genes.

Cancer cells include any cells derived from a tumor, neoplasm, cancer, precancer, cell line, malignancy, or any other source of cells that have the potential to expand and grow to an unlimited degree. Cancer cells may be derived from naturally occurring sources or may be artificially created. Cancer cells may also be capable of invasion into other tissues and metastasis when placed into an animal host. Cancer cells further encompass any malignant cells that have invaded other tissues and/or metastasized. One or more cancer cells in the context of an organism may also be called a cancer, tumor, neoplasm, growth, malignancy, or any other term used in the art to describe cells in a cancerous state.

Examples of cancers that could serve as sources of cancer cells include solid tumors such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Additional cancers that may serve as sources of cancer cells include blood borne cancers such as acute lymphoblastic leukemia (“ALL,”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia, multiple myeloma, lymphoblastic leukemia, myelogenous leukemia, lymphocytic leukemia, myelocytic leukemia, Hodgkin's disease, non-Hodgkin's Lymphoma, Waldenstrom's macroglobulinemia, Heavy chain disease, and Polycythemia vera.

In some aspects of the invention, the cancer cells are derived from non-small cell lung cancer (NSCLC.) NSCLC includes any carcinoma derived from lung tissues that does not include small cell lung cancers. Examples of non-small cell lung cancers include adenocarcinomas, large cell carcinomas, and squamous cell carcinomas of the lung.

The pathologic stages of non-small cell lung cancer include, but are not limited to the following: in the occult or hidden stage, cancer cells may be found in sputum, but no tumor can be found in the lung by bronchoscopy or other imaging. In Stage 0, also called carcinoma in situ, abnormal cells are found in the innermost lining of the lung. Such abnormal cells are precancerous and may or may not become malignant and spread into nearby tissue.

In Stage I, a cancer has developed. There are two substages to stage 1. In Stage IA, the tumor presents only in the lung only and is 3 centimeters or smaller. For the disease to be considered stage 1B, it will have one or more of the following traits: the tumor is larger than 3 centimeters, the cancer has spread to the main bronchus of the lung, and is at least 2 centimeters from the carina, the cancer has spread to the innermost layer of the membrane that covers the lungs, or the tumor partly blocks the bronchus or bronchioles and part of the lung has collapsed or developed pneumonitis (inflammation of the lung).

Similarly, there are two substages to Stage II. In Stage IIA, the tumor is 3 centimeters or smaller and cancer has spread to nearby lymph nodes on the same side of the chest as the tumor. For the disease to be considered, Stage IIB, the cancer has spread to nearby lymph nodes on the same side of the chest as the tumor and it will have one or more of the following traits: the tumor is larger than 3 centimeters, the cancer has spread to the main bronchus of the lung and is 2 centimeters or more from the carina, the cancer has spread to the innermost layer of the membrane that covers the lungs, or the tumor partly blocks the bronchus or bronchioles and part of the lung has collapsed or developed pneumonitis (inflammation of the lung). Alternatively, the disease may be classified as Stage 2B if the cancer has not spread to the lymph nodes and it displays one or more of the following traits: cancer has spread to the chest wall, or the diaphragm, or the pleura between the lungs, or membranes surrounding the heart, the cancer has spread to the main bronchus of the lung and is no more than 2 centimeters from the carina, but has not spread to the trachea, cancer blocks the bronchus or bronchioles and the whole lung has collapsed or developed pneumonitis (inflammation of the lung). Stage III is also divided into two substages.

In stage IIIA, cancer has spread to lymph nodes on the same side of the chest as the tumor and it displays one or more of the following traits: cancer has spread to the main bronchus, the chest wall, the diaphragm, the pleura around the lungs, or the membrane around the heart, but has not spread to the trachea, or part or all of the lung may have collapsed or developed pneumonitis (inflammation of the lung). In stage IIIB, the tumor has spread to one or more of the following: lymph nodes above the collarbone or in the opposite side of the chest from the tumor, to the heart, to major blood vessels that lead to or from the heart, to the chest wall, to the diaphragm, to the trachea, to the esophagus, to the sternum or spine, to more than one area in the same lobe of the lung, or to the fluid of the pleural cavity surrounding the lung.

In stage IV, cancer may have spread to lymph nodes and has spread to another lobe of the lung or to other parts of the body, such as the brain, liver, adrenal glands, kidneys, or bone.

The present invention further provides kits to be used in assessing the expression of a microRNA in a subject to assess the risk of developing disease. Kits include any combination of components that facilitates the performance of an assay. A kit that facilitates assessing the expression of a microRNA may include suitable nucleic acid-based and immunological reagents as well as suitable buffers, control reagents, and printed protocols.

Kits that facilitate nucleic acid based methods may further include one or more of the following: specific nucleic acids such as oligonucleotides, labeling reagents, enzymes including PCR amplification reagents such as Tag or Pfu, reverse transcriptase, or other, and/or reagents that facilitate hybridization.

Specific nucleic acids may include nucleic acids, polynucleotides, oligonucleotides (DNA, or RNA), or any combination of molecules that includes one or more of the above, or any other molecular entity capable of specific binding to a nucleic acid target. In one aspect of the invention, the specific nucleic acid comprises one or more oligonucleotides capable of hybridizing to the target.

A specific nucleic acid may include a label. A label may be any substance capable of aiding a machine, detector, sensor, device, or enhanced or unenhanced human eye from differentiating a sample that that displays positive expression from a sample that displays reduced expression. Examples of labels include but are not limited to: a radioactive isotope or chelate thereof, a dye (fluorescent or nonfluorescent,) stain, enzyme, or nonradioactive metal. Specific examples include but are not limited to: fluorescein, biotin, digoxigenin, alkaline phosphatase, biotin, streptavidin, 3H, 14C, 32P, 35S, or any other compound capable of emitting radiation, rhodamine, 4-(4′-dimethylaminophenylazo) benzoic acid (“Dabcyl”); 4-(4′-dimethylamino-phenylazo)sulfonic acid (sulfonyl chloride) (“Dabsyl”); 5-((2-aminoethyl)-amino)-naphtalene-1-sulfonic acid (“EDANS”); Psoralene derivatives, haptens, cyanines, acridines, fluorescent rhodol derivatives, cholesterol derivatives; ethylenediaminetetraaceticacid (“EDTA”) and derivatives thereof or any other compound that signals the presence of the labeled nucleic acid. In one embodiment of the invention, the label includes one or more dyes optimized for use in genotyping. Examples of such dyes include but are not limited to: dR110, 5-FAM, 6FAM, dR6G, JOE, HEX, VIC, TET, dTAMRA, TAMRA, NED, dROX, PET, and LIZ.

An oligonucleotide may be any polynucleotide of at least 2 nucleotides. Oligonucleotides may be less than 10, 15, 20, 30, 40, 50, 75, 100, 200, or 500 nucleotides in length. While oligonucleotides are often linear, they may assume a circular or other two dimensional structure. Oligonucleotides may be chemically synthesized by any of a number of methods including sequential synthesis, solid phase synthesis, or any other synthesis method now known or yet to be disclosed. Alternatively, oligonucleotides may be produced by recombinant DNA based methods. In some aspects of the invention, an oligonucleotide may be 2 to 1000 bases in length. In other aspects, it may be 5 to 500 bases in length, 5 to 100 bases in length, 5 to 50 bases in length, or 10 to 30 bases in length. One skilled in the art would understand the length of oligonucleotide necessary to perform a particular task. Oligonucleotides may be directly labeled, used as primers in PCR or sequencing reactions, or bound directly to a solid substrate as in oligonucleotide arrays.

A nucleotide is an individual deoxyribonucleotide or ribonucleotide base. Examples of nucleotides include but are not limited to: adenine, thymine, guanine, cytosine, and uracil, which may be abbreviated as A, T, G, C, or U in representations of oligonucleotide or polynucleotide sequence.

In some aspects of the invention, the probe may be affixed to a solid substrate. In other aspects of the invention, the sample may be affixed to a solid substrate. A probe or sample may be covalently bound to the substrate or it may be bound by some non covalent interaction including electrostatic, hydrophobic, hydrogen bonding, Van Der Waals, magnetic, or any other interaction by which a probe such as an oligonucleotide probe may be attached to a substrate while maintaining its ability to recognize the allele to which it has specificity. A substrate may be any solid or semi solid material onto which a probe may be affixed, attached or printed, either singly or in the formation of a microarray. Examples of substrate materials include but are not limited to polyvinyl, polystyrene, polypropylene, polyester or any other plastic, glass, silicon dioxide or other silanes, hydrogels, gold, platinum, microbeads, micelles and other lipid formations, nitrocellulose, or nylon membranes. The substrate may take any form, including a spherical bead or flat surface. For example, the probe may be bound to a substrate in the case of an array. The sample may be bound to a substrate in the case of a Southern Blot.

### Example

Inventors herein report that patients with lower levels of microRNA-520e had significantly improved disease-free survival (DFS) and overall survival (OS). RNA (>70% tumor) was extracted from 169 primary surgically resected NSCLC tumors. Tumor samples were obtained after written informed consent from patients receiving care and follow-up at the Medical University of Gdansk from 2001 to 2004. Patients did not receive chemotherapy or radiotherapy as per standard local practice at the time of surgical resection. Clinical and biologic information (sex, age at diagnosis, surgical date, histology, tumor grade, surgical margin, pathological stage, smoking history, disease-free and overall survival) were obtained. MicroRNA-520e expression level was measured retrospectively.

To determine relative microRNA-520e expression, qRT-PCR was carried out using the Applied Biosystems TaqMan system, followed by quantitation and normalization to RNU6B (housekeeping control) according to manufacturer's instructions (Livak and Schmittgen 2001). Each sample was analyzed in triplicate on each quantitation run.

NSCLC cell lines used included squamous cell carcinoma line, H157, and adenocarcinoma lines, Colo699 and H2122. Lines were maintained in RPMI media supplemented with 10% heat inactivated fetal bovine serum in a humidified incubator with 5% CO₂.

The Alamar Blue assay (Biosource, Camarillo, Calif.) was used to assess proliferation as described in Kita D, Cancer Res 61, 7985-7991 (2001.) Cells were transiently transfected with 100 uM or miR-520e mimic, miR-520e inhibitor, control mimic or control inhibitor. Approximately 1,000 cells of each population for each line were seeded in quadruplicate wells of 96-well plastic plates in 200 μA of culture medium supplemented with 10% fetal bovine serum and. The plates were incubated for 4 hours at 37° C. and Alamar Blue was added in a volume of 20 μl (10% of total volume) to the cells and incubated for 3 hours. The plate was read on a fluorescence plate reader (excitation, 530 nm; emission, 590 nm) at 48 hours (correlating to 96 hours after miR transfections). Averages of the absorbance values were calculated and plotted. Relative cell growth rate per hour was determined relative to number of cells at time zero relative to control. MicroRNA-520e mimic treated cells were normalized to control mimic and microRNA-520e inhibitor treated cells were normalized to control inhibitor.

For cell cycle studies, NSCLC cells were seeded at 5.0×10⁵ cells per 24-mm plate and transiently transfected with 100 μM of microRNA-520e mimic, inhibitor, control mimic, or control inhibitor as previously described. After 144 hr incubation the cells were collected and fixed in cold 70% ethanol (−20° C.) for 2 hrs. After washing twice with PBS, cells were resuspended in PBS. RNase A (0.5 mg/ml) and PI (2.5 μg/ml) were added to the fixed cells for 30 min. The DNA content of cells was then analyzed with a CyAn ADP Analyzer instrument. The mean proliferation index (MPI) was determined by the following equation S+G₂M/[S+G₂M+G₀G₁].

For all statistical tests, results with P<0.05 were considered significant. The Chi-square test or Fisher's exact test was used to analyze concordance/discordance among proportions of two categorical factors under study. The Kaplan-Meier method was used to estimate the probability of survival as a function of time. Disease-free survival was calculated from the date of surgical resection to the date of documented disease relapse; all other patients were censored at the time of their last follow-up. Overall survival was calculated from the date of surgical resection to the date of death from any cause; all other patients were censored at the time of their last follow-up. The survival difference for disease-free or overall survival among patients grouped using single clinical factor was assessed using log-rank test and univariate Cox regression independently. The relative importance of multiple clinical factors to disease-free or overall survival was assessed using the Cox proportional-hazards method.

Comparison of messenger RNA microarray data on NSCLC cell lines revealed a subset of highly over expressed genes in NSCLC cell lines that were predicted to be regulated by the same microRNA, microRNA-520. The chromosomal location of this microRNA was examined and a cluster of similar microRNAs was discovered on chromosome 19q13, a region of the genome commonly altered in lung cancer. The microRNAs in the microRNA-520 cluster (520a, 520b, 520c, 520d, 520e, 520f, 520g, 520h) all share the same predicted binding seed sequence, thus would be expected to target the same set of mRNAs.

We retrospectively assessed microRNA-520e levels by qRT-PCR in 169 samples from primary surgically resected NSCLC tumors obtained with written informed consent from patients receiving care and follow-up at the Medical University of Gdansk. Clinical and biologic information (sex, age at diagnosis, surgical date, histology, tumor grade, surgical margin, pathological stage, smoking history, DFS and OS) were obtained (n=169), only type of surgical resection and pathologic stage were significantly associated with DFS and OS. There were no statistically significant differences in DFS or OS amongst the pathologic subtypes, age, gender, or tumor grade (Table II). Type of surgical resection resulted in a statistically significant DFS and OS (p=0.0057 and 0.0019, respectively) as expected, with pneumonectomy showing worse outcome compared to lobectomy or bilobectomy.

The comparison by pathological stages (I-IIIA) resulted in statistically significant differences in both DFS and OS (p=5.62×10⁻⁷ and 7.93×10⁻⁸, respectively), where earlier stage was associated with better prognosis. Patients who had positive surgical margins had significantly shorter DFS (p=0.0394) but not OS (p=0.097).

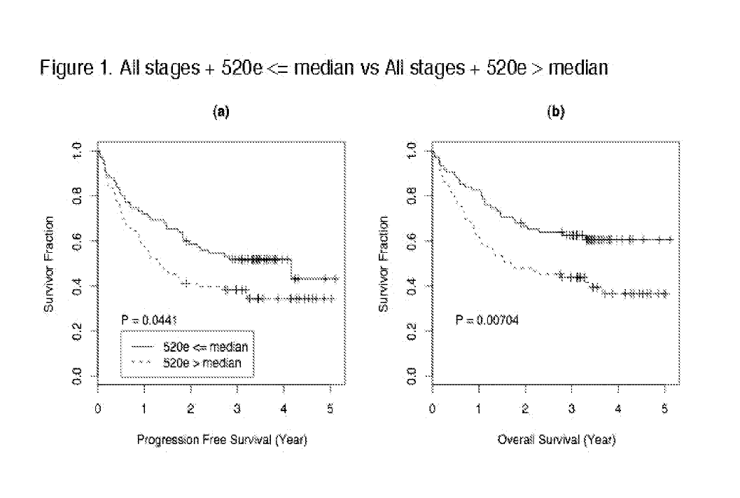

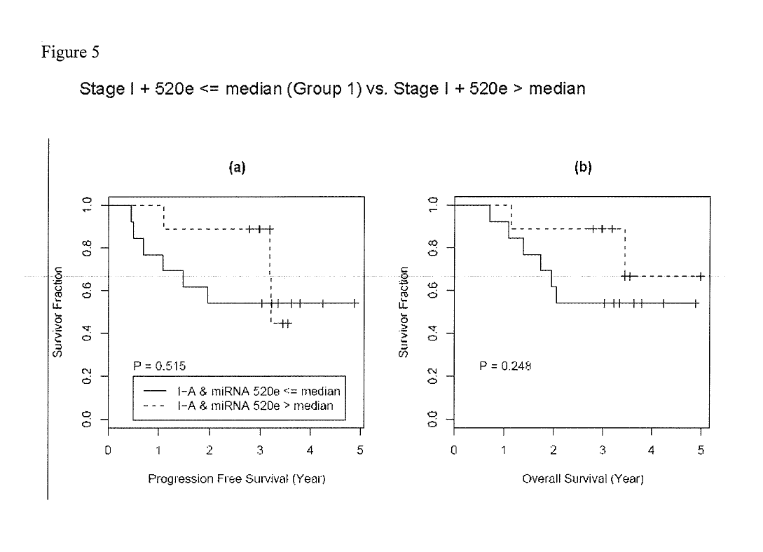

Measurement of microRNA-520e expression (n=148) revealed that reduced levels (below the population median) were significantly associated with improved DFS (p=0.04) and OS (p=0.007) (FIG. 1). Reduced microRNA-520e DFS was 4.2 years and OS was not reached, compared to those with positive microRNA-520e, DFS and OS was 1.3 and 1.7 years, respectively. Comparing by pathologic stage, reduced microRNA-520e expression significantly correlated with improved OS for stage III (p=0.037) and favors DFS and OS for the other stages (FIGS. 5-9 and Tables 5-9). The median DFS and OS has not been reached for low microRNA-520e in stage I and II patients. Median microRNA-520e expression in the population was 9.07×10⁻⁶ relative to expression of RNU6B by QRTPCR. This expression corresponded to 34-39 PCR cycles to reach a predetermined optical density of bound double stranded DNA binding dye (SYBR Green). Expression below the median level was generally undetectable—dye incorporation failed to reach the predetermined level even after 40 cycles.

By multivariate analysis, DFS was significantly associated with gender, pathologic stage and microRNA-520e expression (Hazard ratio [HR] 2.03, 95% confidence interval [CI] 1.03-4.00; HR 1.96, 95% CI 1.43-2.70; and HR 1.75, 95% CI 1.08-2.82) (Table 3). By multivariate analysis, OS was significantly associated with both pathologic stage and microRNA-520e expression (HR 2.04, 95% CI 1.48-2.81 and HR 1.89, 95% CI 1.17-3.06, respectively) (Table 4).

SKY and CGH were used to confirm that chromosome loss or rearrangement of this region on 19q13 was a frequent event in NSCLC cell lines. H157, one of three NSCLC cell lines showed a rearrangement involving 19q13. In the H157 line, microRNA-520e expression was consistently reduced (⅓ to nearly 4½ fold less) relative to the other two NSCLC lines, which had no chromosome 19q13 rearrangements.

Ingenuity Pathway Analysis (IPA) was used to determine if the loss of microRNA-520e would be expected to regulate central pathways involving multiple proteins. The list of predicted targets of microRNA-520e to IPA of predicted targets of microRNA-520e revealed only one significant pathway, G1/S Checkpoint Regulation. Based on this result, the effect of microRNA-520e mimic and inhibitor transfection on cellular proliferation, cellular migration, cell cycle, and apoptosis was assessed.

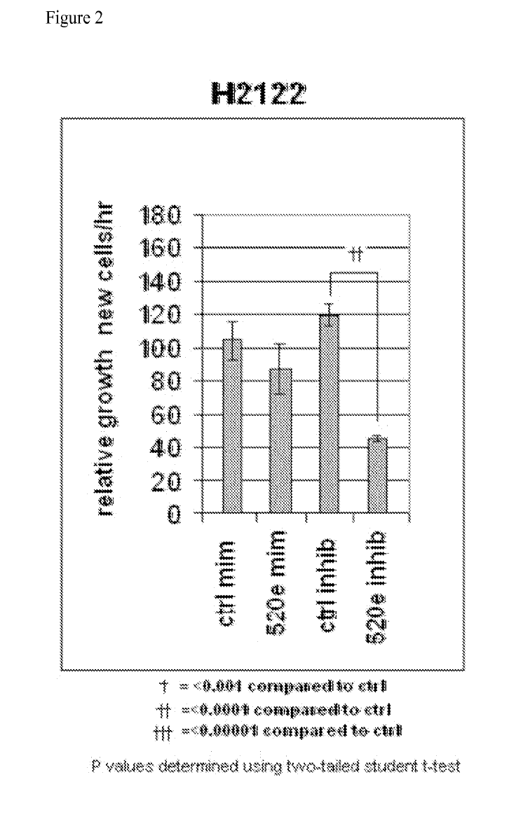

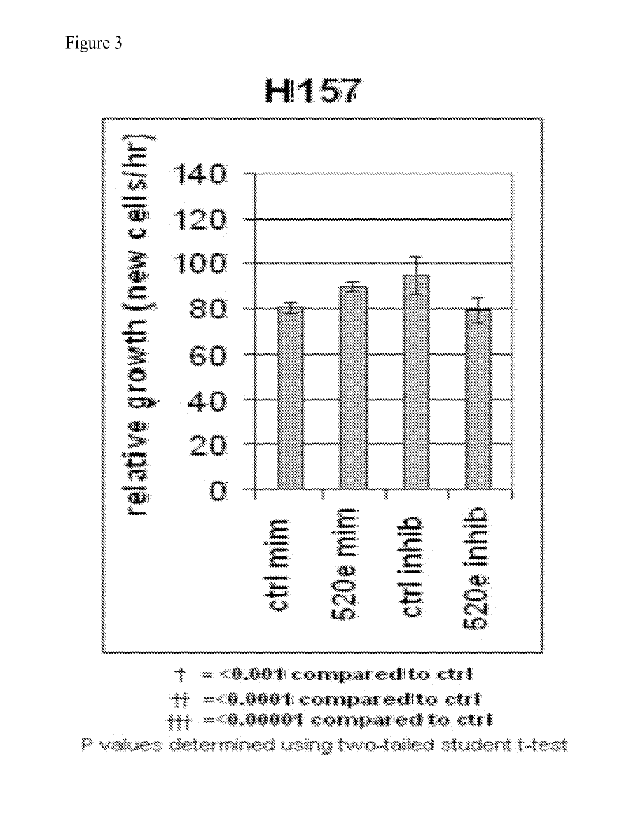

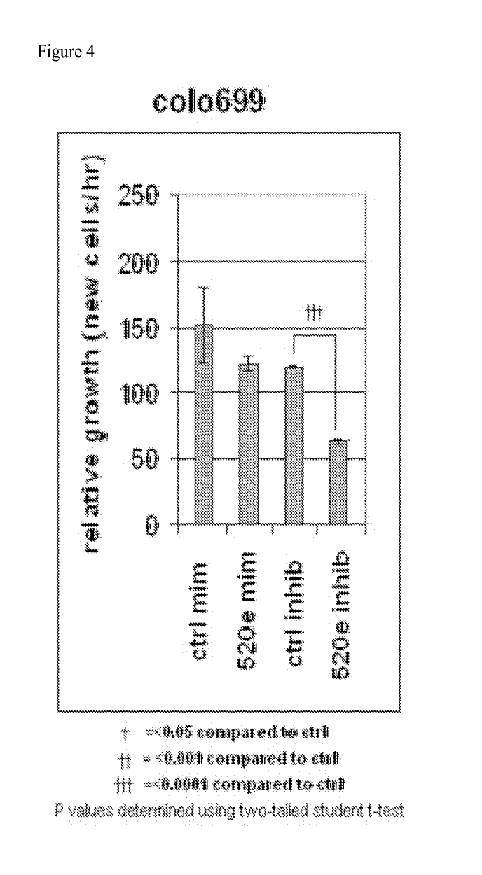

A significant decrease in cell proliferation was observed with microRNA mimic in H2122 and Colo699 (˜2-3 fold), while no significant alterations were seen with microRNA-520e inhibitor (FIGS. 2-4). H157, did not show decreased proliferation in response to microRNA-520e inhibition, likely in part because microRNA-520e expression is relatively low, and the alteration at 19q13 may have resulted in an alternative pathway driving proliferation. Mimic transfection increased the mean proliferation index (MPI) in H157 relative to control (cell line with lowest basal microRNA-520e expression). Neither microRNA-520e mimic nor inhibitor had an effect on cell migration or apoptosis.

### REFERENCES

The following and all patents, patent applications, and publications above are hereby incorporated by reference in their entirety.
1. Potti A et al, N Engl J Med 355, 570-580 (2006).
2. Sanchez-Cespedes M et al, Cancer Res 15, 1309-1313 (2001).
3. The microRNA Registry, http://microrna.sanger.ac.uk/sequences/
4. Huang Q et al, Nat Cell Biol 10, 501-502 (30 Apr. 2008.)
5. Rosenfeld N et al, Nat Biotechnol 26, 462-46 (23 Mar. 2008.)
6. Yu S L et al, Cancer Cell 13, 48-57 (1 Jan. 2008.)
7. Livak and Schmittingen, Methods 25, 402-408, 2001.

### TABLES

TABLE 1
Description of samples used in the study

| Parameter¹ | Summary |
|---|---|
| Age (169) | (37.18, 64.08, 62.56, 85.25)² |
| | <70: 130, ≧70: 39 |
| Gender (169) | Female: 42, Male: 127 |
| Smoking (169) | Never: 8, Ever: 161 |
| Surgery type (169) | Wedge: 1, Segmentectomy: 2, Lobectomy: 89, |
| | Bilobectomy: 10, Pneumonectomy: 61, Sleeve |
| | lobectomy: 6 |
| Pathology (169) | SCC: 96, AC: 48, LCC: 2, NSCLC |
| | NOS/MIXED: 22, NSCLC OTHER: 1 |
| Pathologic Stage (169) | IA: 24, IB: 51, IIA: 4, IIB: 38, IIIA: 52 |
| Grade (146) | G1: 17, G2: 73, G3: 56 |
| Remains (152) | Complete: 137, Microscopic positive margin: 14, |
| | Macroscopic positive margin: 1 |

¹numbers in parentheses represent the number of non-missing cases.
²these numbers represent minimum, median, mean, maximum values.

TABLE 2
Further classification of samples used in study

| Characteristics | | N | PFS (years)¹ | OS (years)² |
|---|---|---|---|---|
| Age group | <70 | 130 | (0.04, 0.64, 0.98, 4.16)³ | (0.04, 0.85, 1.01, 3.66) |
| | ≧70 | 39 | (0.03. 0.6, 0.9, 2.72) | (0.03, 0.89, 1.01, 3.28) |
| | P⁴ | | 0.795 | 0.601 |
| Gender | Female | 42 | (0.05, 0.48, 0.86, 3.67) | (0.05, 0.7, 0.62, 1.29) |
| | Male | 127 | (0.03, 0.73, 0.98, 4.16) | (0.03, 0.91, 1.1, 3.66) |
| | P | | 0.246 | 0.157 |
| Surgery | Lobectomy | 89 | (0.06, 0.89, 1.17, 3.67) | (0.06, 0.9, 1.26, 3.66) |
| | Bilobectomy | 10 | (0.87, 1.36, 1.39, 1.96) | (1.06, 1.69, 1.83, 1.96) |
| | Pneumonectomy | 61 | (0.03, 0.5, 0.7, 4.16) | (0.03, 0.54, 0.72, 1.78) |
| | P | | 0.00572 | 0.00192 |
| Pathology | SCC | 96 | (0.04, 0.58. 0.93, 4.16) | (0.04, 0.9, 1.04, 3.66) |
| | AC | 48 | (0.03, 0.87, 1.13, 3.21) | (0.03, 0.86, 1.11, 3.29) |
| | NSCLC NOS/MIXED | 24 | (0.07, 0.76, 0.93, 2.2) | (0.07, 0.73, 0.75, 1.76) |
| | P | | 0.155 | 0.219 |
| Pathologic | I (IA + IB) | 75 | (0.09, 0.86, 1.18, 3.21) | (0.09, 1.08, 1.36, 3.44) |
| Stage | II (IA + IIB) | 42 | (0.06, 1.03, 1.06, 3.67) | (0.06, 1.19, 1.22, 3.66) |
| | III (IIIA) | 52 | (0.03, 0.52, 0.74, 4.16) | (0.03, 0.58, 0.66, 1.78) |
| | P | | 5.62 × 10⁻⁷ | 7.93 × 10⁻⁸ |
| Grade | G1 | 17 | (0.09, 0.89, 1.25, 3.67) | (0.09, 1.1, 1.06, 2.29) |
| | G2 | 73 | (0.03, 0.86, 1.02, 3.21) | (0.03, 1.01, 1.12, 3.44) |
| | G3 | 29 | (0.04, 0.62, 0.96, 4.16) | (0.04, 0.71, 1.03, 3.66) |
| | P | | 0.721 | 0.841 |
| Remains | Complete | 137 | (0.03, 0.77. 0.98, 3.67) | (0.03, 0.89, 1.11, 3.66) |
| | Microscopic positive | 14 | (0.05, 0.53, 1.06, 4.16) | (0.05, 0.53, 0.69, 1.69) |

| | | |
|---|---|---|
| P | 0.039 | 0.097 |

¹calculated over relapsed samples only. (Non-relapsed samples were filtered out.)
²calculated over dead samples only. (Samples that were still alive at the time of last follow-up were filtered out.)
³quartet combinations of numbers in parentheses represent minimum, median, mean, maximum values.
⁴survival difference p values are calculated using the log-rank test.

TABLE 3
Disease-free survival Multivariate Analysis

| | | | 95% Confidence | |
|---|---|---|---|---|
| | | Relative | Interval | |
| Variable | P-value | Hazard | Lower | Upper |
| Age group | 0.56 | 1.20 | 0.65 | 2.21 |
| Gender | 0.04 | 2.03 | 1.03 | 4.00 |
| Surgery | 0.66 | 1.06 | 0.83 | 1.36 |
| Pathologic | 0.000033 | 1.96 | 1.43 | 2.70 |
| stage | | | | |
| Remains | 0.28 | 1.44 | 0.75 | 2.76 |
| MicroRNA- | 0.02 | 1.75 | 1.08 | 2.82 |
| 520e | | | | |

Hypothesis testing results (H0: all regression coefficients = 0)

| | | |
|---|---|---|
| Likelihood ratio test | 37.2 on 6 df | P-value = 1.63 · 10⁻⁶ |
| Wald test | 34.3 on 6 df | P-value = 5.81 × 10⁻⁶ |
| Log-rank test | 37.4 on 6 df | P-value = 1.48 × 10⁻⁶ |

TABLE 4
Multivariate Analysis - Overall Survival

| | | | 95% Confidence | |
|---|---|---|---|---|
| | | Relative | Interval | |
| Variable | P-value | Hazard | Lower | Upper |
| Age group | 0.33 | 1.01 | 0.99 | 1.04 |
| Gender | 0.05 | 1.99 | 1.00 | 3.95 |
| Surgery | 0.59 | 1.07 | 0.84 | 1.38 |
| Pathologic | 0.000014 | 2.04 | 1.48 | 2.81 |
| stage | | | | |
| Remains | 0.27 | 1.45 | 0.75 | 2.82 |
| microRNA- | 0.009 | 1.89 | 1.17 | 3.06 |
| 520e | | | | |

Hypothesis testing results (H0: all regression coefficients = 0)

| | | |
|---|---|---|
| Likelihood ratio test | 41.3 on 6 df | P-value = 2.5 · 10⁻⁷ |
| Wald test | 37.6 on 6 df | P-value = 1.34 × 10⁻⁶ |
| Log-rank test | 41.3 on 6 df | P-value = 2.53 × 10⁻⁷ |

TABLE 5
Disease-free and overall survival based on miR-520e expression. All
pathologic stages included. Group 1 is miR-520e expression ≦ median
for all samples, Group 2 is miR-520e expression > median for all samples
(See FIG. 1).
Disease-free survival
Cox regression test (likelihood test p = 0.0448; Wald test p = 0.0458;
Log-rank test p = 0.0441)

| Regression | Standard | Wald | | Relative | | |
|---|---|---|---|---|---|---|
| Coefficient | Error | Statistic | p-vlaue | Hazard | Low CI | High CI |
| 0.44 | 0.22 | 2 | 0.046 | 1.55 | 1.01 | 2.39 |

median survival for group1: 4.16 (y)
median survival for group2: 1.33 (y)

overall survival
Cox regression test (likelihood test p = 0.00721;
Wald test p = 0.00803; Log-rank test p = 0.00704)

| Regression | Standard | Wald | | Relative | | |
|---|---|---|---|---|---|---|
| Coefficient | Error | Statistic | p-vlaue | Hazard | Low CI | High CI |
| 0.635 | 0.24 | 2.65 | 0.008 | 1.89 | 1.18 | 3.02 |

median survival for group1: NA
median survival for gorup2: 1.69 (y)

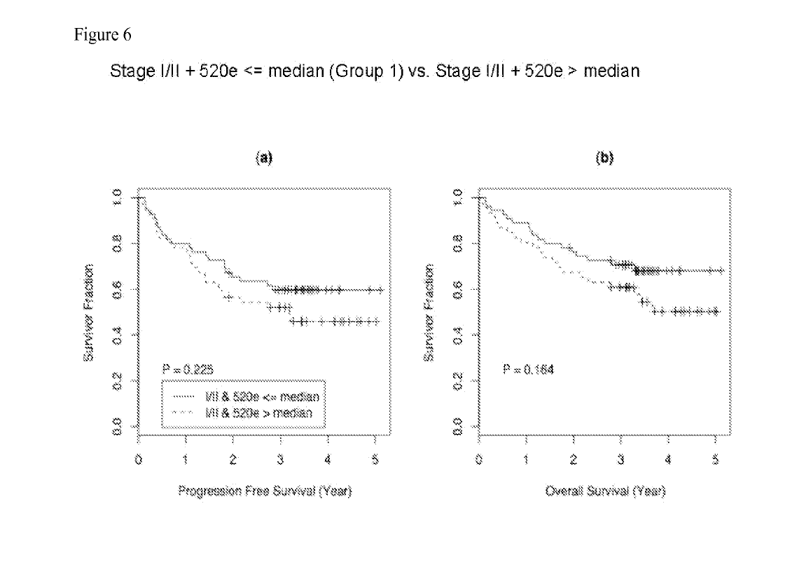

TABLE 6
Disease-free and overall survival based on miR-520e expression.
Pathologic Stage I only. Group 1 is miR-520e expression ≦ median
for all samples, Group 2 is miR-520e expression > median for all
samples (See FIG. 6).
Disease-free survival
Cox regression test (likelihood test p = 0.228;
Wald test p = 0.228; Log-rank test p = 0.225)

| Regression | Standard | Wald | | Relative | | |
|---|---|---|---|---|---|---|
| Coefficient | Error | Statistic | p-vlaue | Hazard | Low CI | High CI |
| 0.356 | 0.295 | 1.21 | 0.23 | 1.43 | 0.8 | 2.55 |

median survival for group1: NA
median survival for group2: 3.19 (y)

overall survival
Cox regression test (likelihood test p = 0.166;
Wald test p = 0.168; Log-rank test p = 0.164)

| Regression | Standard | Wald | | Relative | | |
|---|---|---|---|---|---|---|
| Coefficient | Error | Statistic | p-vlaue | Hazard | Low CI | High CI |
| 0.399 | 0.327 | 1.22 | 0.22 | 1.49 | 0.786 | 2.83 |

median survival for group1: NA
median survival for gorup2: 3.66 (y)

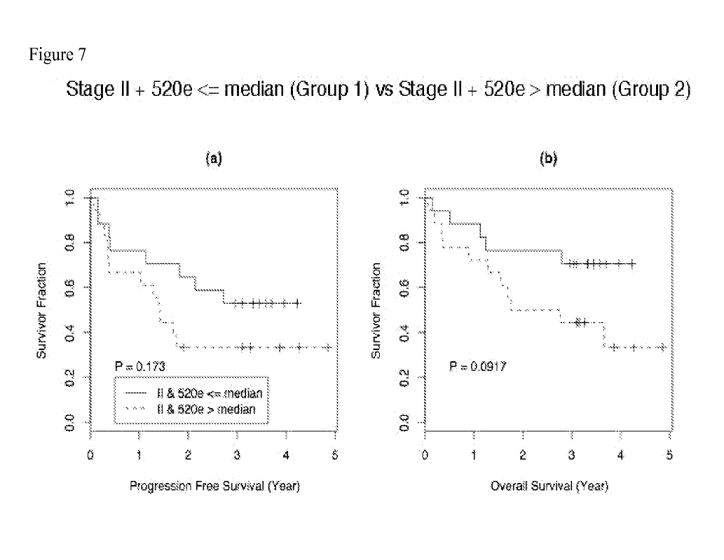

TABLE 7
Disease-free and overall survival based on miR-520e expression.
Pathologic Stages I and II included. Group 1 is miR-520e
expression ≦ median for all samples, Group 2 is miR-520e
expression > median for all samples (See FIG. 7).
Disease-free survival
Cox regression test (likelihood test p = 0.174;
Wald test p = 0.180; Log-rank test p = 0.173)

| Regression | Standard | Wald | | Relative | | |
|---|---|---|---|---|---|---|
| Coefficient | Error | Statistic | p-vlaue | Hazard | Low CI | High CI |
| 0.618 | 0.461 | 1.34 | 0.18 | 1.85 | 0.752 | 4.57 |

median survival for group1: NA
median survival for group2: 1.4 (y)

overall survival
Cox regression test (likelihood test p = 0.0889;
Wald test p = 0.102; Log-rank test p = 0.0917)

| Regression | Standard | Wald | | Relative | | |
|---|---|---|---|---|---|---|
| Coefficient | Error | Statistic | p-vlaue | Hazard | Low CI | High CI |
| 0.884 | 0.541 | 1.63 | 0.1 | 2.42 | 0.838 | 6.99 |

median survival for group1: NA
median survival for gorup2: 1.76 (y)

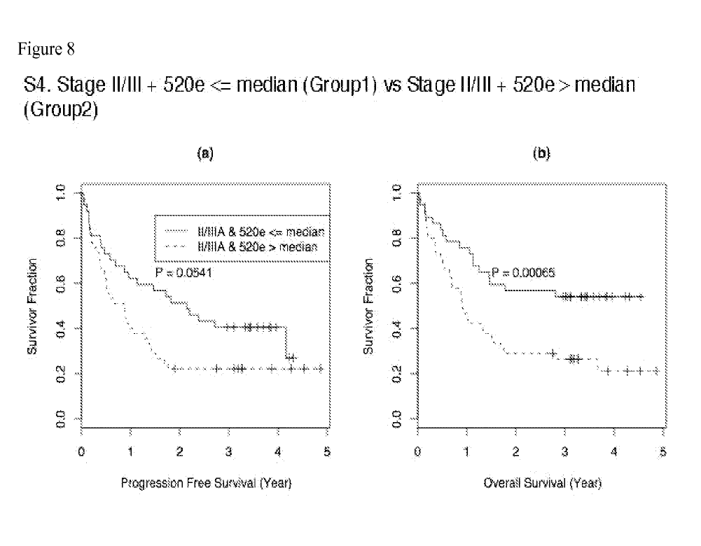

TABLE 8
Disease-free and overall survival based on miR-520e expression.
Pathologic Stage II only. Group 1 is miR-520e expression ≦ median
for all samples, Group 2 is miR-520e expression > median
for all samples (See FIG. 8).
Disease-free survival
Cox regression to (likelihood test p = 0.0537;
Wald test p = 0.0567; Log-rank test p = 0.0541)

| Regression | Standard | Wald | | Relative | | |
|---|---|---|---|---|---|---|
| Coefficient | Error | Statistic | p-vlaue | Hazard | Low CI | High CI |
| 0.517 | 0.271 | 1.91 | 0.057 | 1.68 | 0.985 | 2.85 |

median survival for group1: 2.14 (y)
median survival for group2: 0.87 (y)

overall survival
Cox regression test (likelihood test p = 0.00615;
Wald test p = 0.00795; Log-rank test p = 0.0065)

| Regression | Standard | Wald | | Relative | | |
|---|---|---|---|---|---|---|
| Coefficient | Error | Statistic | p-vlaue | Hazard | Low CI | High CI |
| 0.793 | 0.299 | 2.65 | 0.0079 | 2.21 | 1.23 | 3.97 |

median survival for group1: NA
median survival for gorup2: 0.92 (y)

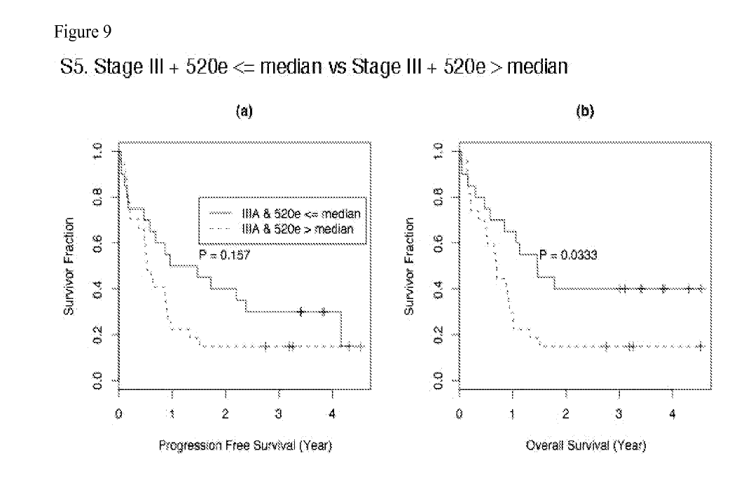

TABLE 9
Disease-free and overall survival based on miR-520e expression.
Pathologic Stages II and III combined. Group 1 is miR-520e
expression ≦ median for all samples, Group 2 is miR-520e
expression > median for all samples (See FIG. 9).
Disease-free survival
Cox regression test (likelihood test p = 0.156;
Wald test p = 0.161; Log-rank test p = 0.157)

| Regression | Standard | Wald | | Relative | | |
|---|---|---|---|---|---|---|
| Coefficient | Error | Statistic | p-vlaue | Hazard | Low CI | High CI |
| 0.473 | 0.337 | 1.4 | 0.16 | 0.16 | 0.829 | 3.11 |

median survival for group1: 0.96 (y)
median survival for group2: 0.53 (y)

overall survival
Cox regression test (likelihood test p = 0.0322;
Wald test p = 0.0372; Log-rank test p = 0.0333)

| Regression | Standard | Wald | | Relative | | |
|---|---|---|---|---|---|---|
| Coefficient | Error | Statistic | p-vlaue | Hazard | Low CI | High CI |
| 0.755 | 0.363 | 2.08 | 0.037 | 2.13 | 1.05 | 4.33 |

median survival for group1: 1.47 (y)
median survival for gorup2: 0.7 (y)

TABLE 10
Relative microRNA expression in NSCLC cell lines

| Assessment | H2122 | H157 | Colo699 |
|---|---|---|---|
| Histology | Adeno | SCC | Adeno |
| Chromsome 19q13 | No | Yes | No |
| rearrangement | | | |
| Relative MicroRNA-520e* | 3.1 | 0.7 | 1 |

Key: SCC= squamous cell carcinoma;
Adeno = adeno-carcinoma;
IHC = immunohistochemistry;
* = MicroRNA-520e normalized to RNU6B, relative to Colo699 expression

We claim:

1. A method of predicting a reduced risk of recurrence of non-small cell lung cancer in a subject comprising:
obtaining a sample from the subject;
assessing the expression of a target selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8;
comparing the expression of the target in the sample with an expression level predetermined to correlate with reduced risk of recurrence.

2. The method of claim 1 wherein the sample comprises a tumor cell.

3. The method of claim 2 wherein the sample comprises sputum.

4. The method of claim 2 wherein the tumor cell has a chromosomal aberration in 19q13.

5. The method of claim 1 wherein the sample comprises serum.

6. The method of claim 1 wherein the sample comprises plasma.

7. The method of claim 1 wherein the sample comprises whole blood.

8. The method of claim 1 wherein assessing the expression comprises microarray analysis.

9. The method of claim 1 wherein assessing the expression comprises RTPCR.

10. The method of claim 1 wherein assessing the expression comprises direct sequencing of cDNA.

11. The method of claim 1 wherein the reduced risk comprises increased disease free survival.

12. The method of claim 1 wherein the reduced risk comprises improved overall survival.

13. The method of claim 1 further comprising assessing a characteristic selected from the group consisting of gender and disease stage.

14. The method of claim 13 wherein the characteristic comprises disease stage and the pathologic stage is selected from the group consisting of Stage II and Stage III.

15. The method of claim 1 wherein the expression level comprises any detectable expression of the target.

16. The method of claim 1 wherein the expression level comprises the median expression level in a group comprising at least 100 small cell lung cancer tumors.

17. The method of claim 9 wherein the expression level comprises dye incorporation to a preset optical density within 40 PCR cycles.

18. The method of claim 1 wherein the expression level is about 1×10⁻⁵ relative to expression of a housekeeping gene.

19. A kit to be used in predicting a risk of recurrence of non-small cell lung cancer in a patient comprising:
a first nucleic acid that hybridizes to a target selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

20. The kit of claim 19 wherein the first nucleic acid comprises a fluorescent label.

21. The kit of claim 20 wherein the fluorescent label is selected from the group consisting of FAM, dR110, 5-FAM, 6FAM, dR6G, JOE, HEX, VIC, TET, dTAMRA, TAMRA, NED, dROX, PET, and LIZ.

22. The kit of claim 19 wherein the first nucleic acid is affixed to a solid substrate.

23. The kit of claim 19 further comprising a second nucleic acid that hybridizes to a target selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8, wherein the second nucleic acid does not overlap the sequence to which the first nucleic acid hybridizes.

24. The kit of claim 23 wherein the kit further comprises reverse transcriptase.

25. The kit of claim 23 wherein the kit further comprises a DNA polymerase.
